(19) United States
(12) Patent Application Publication
(10) Pub. No.: US 2026/0031239 A1
(43) Pub. Date: Jan. 29, 2026

(54) Systems and Methods for Querying an Executable Condition Stored in a Database

(71) Applicant: Veeva Systems Inc., Pleasanton, CA (US)

(72) Inventors: Marius K. Mortensen, Burlington (CA); Raagi Pandya, Montreal (CA); Yangyang Xu, Oakville (CA)

(73) Assignee: Veeva Systems Inc., Pleasanton, CA (US)

(21) Appl. No.: 18/785,129

(22) Filed: Jul. 26, 2024

(51) Int. Cl. G16H 50/70; G06F 16/2455

(52) U.S. Cl. CPC G16H 50/70; G06F 16/24564

(57) ABSTRACT

A method for querying and executing a second rule of a first database includes receiving a source file including first medical product data. The method further includes querying a first database to select a first rule. The first rule includes a first rule criteria. The method further includes determining the first medical product data of the source file does not fulfill the first rule criteria. The method further include querying the first database of the provider computing system to select the second rule. The second rule includes a second rule criteria and a target medial product. The method further includes determining the first medical product data of the source file fulfills the second rule criteria. The method further includes querying a second database to select second medical product data associated with the target medical product. The method further includes generating a case dataset and outputting the case dataset.

### TECHNICAL FIELD

The present disclosure relates to systems and methods for querying an executable condition (also referred to as a rule) stored in a database.

### BACKGROUND

Researchers, scientists, industry players, academics, government regulators, and other stakeholders are increasingly in need of efficient and simple ways to retrieve and execute conditions stored in databases.

### SUMMARY

One embodiment relates to a method for querying and executing a second rule of a first database. The method includes receiving a source file including first medical product data. The method further includes querying a first database of the provider computing system to select a first rule. The first rule includes at least one first rule criterion. The method further includes determining the first medical product data of the source file does not fulfill the at least one first rule criterion. The method further include querying, in response to the first medical product data of the source file not fulfilling the at least one first rule criterion, the first database of the provider computing system to select the second rule. The second rule includes at least one second rule criterion and a target medial product. The method further includes determining the first medical product data of the source file fulfills the at least one second rule criterion. The method further includes querying, in response to the first medical product data of the source file fulfilling the at least one second rule criterion, a second database of the provider computing system to select second medical product data associated with the target medical product. The method further includes generating a case dataset including at least a portion of the second medical product data and outputting the case dataset.

Another embodiment relates to a method for querying and executing a first rule of a first database. The method includes receiving a source file including first medical product data. The first medical product data includes a medical product alias. The method further includes querying a first database of the provider computing system to select a ruleset in response to the ruleset including the medical product alias. The ruleset includes at least the first rule and a second rule. The first rule includes at least one rule criterion and a target medical product The method further includes determining the first medical product data of the source file fulfills the at least one rule criterion. The method further includes querying, in response to the first medical product data of the source file fulfilling the at least one rule criterion, a second database of the provider computing system to select second medical product data associated with the target medical product. The method further includes generating a case dataset including at least a portion of the second medical product data and outputting the case dataset.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE FIGURES

FIG. 1 may be a component diagram of a query generation and rule execution system, according to an example embodiment.

FIG. 2 may be a flow diagram of a method for receiving and storing medical product data and rules, according to an example embodiment.

FIG. 3 may be a flow diagram of a method for querying and executing a rule to code a medical product, according to an example embodiment.

FIG. 4 may be an illustration of some aspects of a query generation and rule execution system of FIG. 1 to manage rules, according to an example embodiment.

### DETAILED DESCRIPTION

Referring generally to the figures, systems and methods querying an executable condition stored in a database are disclosed. The systems and methods described herein provide for automatic adverse event processing, medical product coding, and case dataset generation, and thereby help and improve the pharmacovigilance industry by more accurately and more quickly tracking, capturing, and reporting adverse events. For example, by collaborating with a trusted computer system or device for the intake of source files, the present systems can speed up adverse event intake and processing and bypass any manual steps (e.g., medical product coding) in the case dataset generation process, thereby shortening the time it takes for adverse events to be processed, case datasets to be generated and reported to the relevant health authorities. For instance, because the present systems and methods receive medical product verification rules including rule criteria and a target medical product, the present systems and methods can automatically verify medical products thereby not requiring manual correction and checks. Further, because the systems and methods described herein remove time-consuming and laborious manual checks of the medical products and generated case data, the present systems use less computing power and memory than typical case generation systems by not communicating with the user computing device for medical product verification, corrections of the adverse event data, and the like, which also allows for quicker and more efficient case dataset generation and reporting.

Additionally, by utilizing rules to determine a target medical product based on an alias, the present systems and methods provide for increased consistency and accuracy, as well as increased flexibility and scalability. For instance, by utilizing rules to determine target medical products based on an alias, the present systems and methods can enforce specific coding logic, reducing errors and inconsistencies that can arise from manual coding. This is crucial for aliases, which can be confusing and lead to inaccurate reporting. For example, aliases with similar names or slight variations can easily be confused and mixed up. In comparison, rules can define clear criteria for identifying aliases based on factors like product description, dosage information, route of administration, or active ingredients. This eliminates guesswork and ensures consistent coding even with complex aliases. Likewise, by using standardized rules, the present systems and methods ensure all users code the alias of a medical product the same way, improving data integrity and facilitating analysis. Similarly, by utilizing rules to determine a target medical product based on an alias, the present systems and methods be easily adapted to handle new or updated product aliases, ensuring the coding system remains current. For instance, well-designed rule sets can handle a wide range of medical products and their associated aliases, regardless of complexity. This can be especially beneficial for organizations dealing with a large and ever-growing inventory of medical supplies and devices. In comparison, by relying on a flexible rule-based system, the present systems and methods can be updated dynamically to reflect changes in coding standards or regulatory requirements, thereby ensuring the coding process remains compliant and reflects the latest best practices.

As used herein, the term “event,” “medical event,” or “adverse event” can include any untoward medical occurrence which happens to either a patient or a subject in a clinical investigation or during regular use of a medical product that has been given to that person. For example, the “event,” “medical event,” or “adverse event” may encompass any signs which are unfavorable and unexpected for the patient or subject, including any abnormal laboratory findings such as a high blood pressure, a rapid heart rate, etc. The “event,” “medical event,” or “adverse event” could be symptoms, or a disease temporally associated with the use of a medical product and does not have to have been previously associated with that product. The term “event,” “medical event,” or “adverse event” can further encompass adverse reactions and serious adverse events such as death, life-threatening adverse experiences, inpatient hospitalization, congenital birth defects, disabilities, etc. Further, each “event,” “medical event,” or “adverse event” may be defined by the Medical Dictionary for Regulatory Activities (MedDRA) (or other medical code dictionaries) and associated with a specific MedDRA code. Moreover, “event information” “medical event information” “adverse event information” “event data” “medical event data” or “adverse event data” can include information associated with the event such as the date of onset of the event, the date of cessation of the event, the type of event, the dictionary (i.e., digital dictionary, medical dictionary, digital medical dictionary, etc.) or medical term (e.g., MedDRA term), the dictionary or medical code (e.g., MedDRA code), event comments, the outcome of the event, the location of the event (e.g., country where the event occurred), the event duration, patient data for a patient who endured or to which the event occurred, medical products (and associated medical product data (and medical product aliases or tradenames)) or substances that the patient consumed and/or doses for the consumed medical products, the event rank, event contacts, the event type, and any associated event documents.

As used herein, the term “case” or “case dataset” can include an Individual Case Safety Report (ICSR) as defined by the standard ISO/HL7 27953 of the International Standards Organization (ISO) as well as any past or future standards governing ICSRs of the ISO, the World Health Organization (WHO), the Food and Drug Administration (FDA), the European Medicines Agency (EMA), or other national health agencies governing ICSRs. Moreover, “case information” “case data” or “case dataset” can include information associated with or included in the case such as adverse event data, case contact data, case identifier (e.g., case worldwide ID (WWID), case number, etc.), case priority data, case seriousness data, case documents, medical product data (e.g., substances in the medical product, medical product registrations, medical product doses, medical product name, etc.) patient data, and other data associated with a case as defined by the standard ISO/HL7 27953 as well as any past or future standards governing ICSRs of the ISO, the WHO, the FDA, the EMA, or other national health agencies governing ICSRs.

As used herein, the term “substance” can include a substance as defined by the FDA or the EMA. Further, the term “substance” can include an active ingredient or any component of a medical product that provides pharmacological activity or other direct effect in the diagnosis, cure, mitigation, treatment, or prevention of disease, or to affect the structure or any function of the body of man or animals. In this regard, the “substance” may be component responsible for the activity of a medical product.

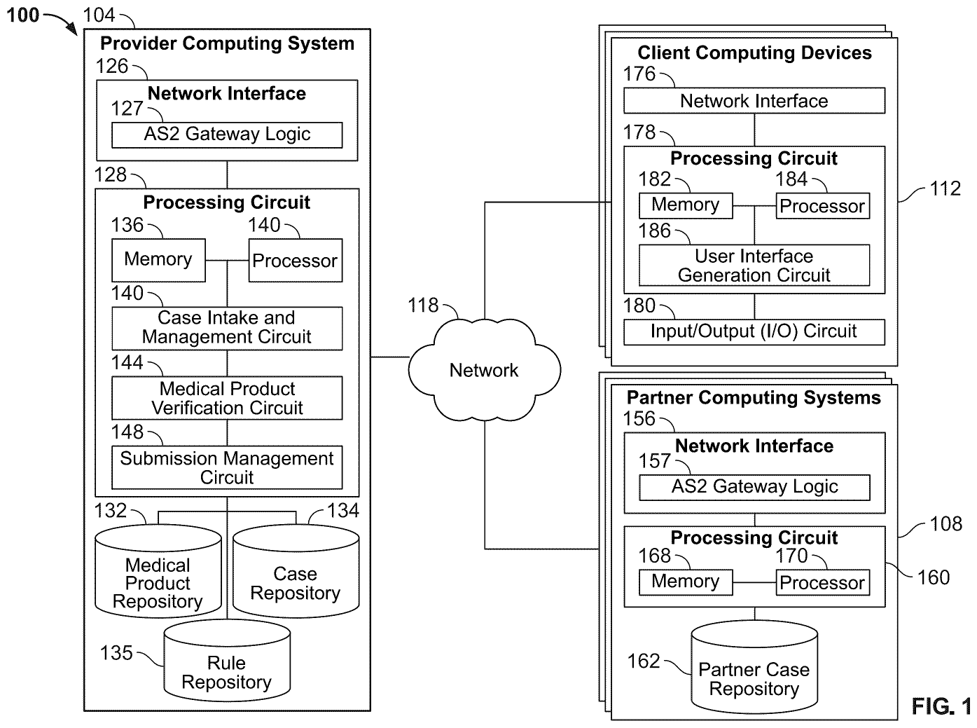

Referring now to FIG. 1, a system 100 for querying and executing a rule is shown, according to an example embodiment. The system 100 includes a provider computing system 104, multiple partner computing systems 108, and multiple client computing devices 112 connected by a secure network (e.g., a network 118).

The network 118 communicably and operably couples the provider computing system 104, the partner computing devices 108, and the client computing devices 112 such that communicable and operable computing may be provided between the provider computing system 104, the partner computing devices 108, and the client computing devices 112 over the network 118. In various embodiments, the network 118 includes any combination of a local area network (LAN), an intranet, the Internet, or any other suitable communications network, directly or through another interface.

The provider computing system 104 may be operated and managed by a provider (e.g., a software as a service (SaaS) provider, a cloud services provider, a software provider, a service provider, etc.) and may include a computer system (e.g., one or more servers (e.g., a cloud computing server) each with one or more processing circuits). In some embodiments, the provider computing system 104 may act as a host and provide an application (e.g., a web-based application, a mobile application, etc.) to the client computing devices 112 over the network 118 in response to authenticating the respective computing device. For example, the provider computing system 104 may receive authentication data (e.g., a username and corresponding password, a limited-use key, a two-factor authentication code or key, etc.) from one of the client computing devices 112. The provider computing system 104 may then authenticate the client computing device 112 based on the authentication data and provide an application to the client computing device 112 over the network 118. In some examples, the provider computing system 104 may be a multi-tenant system where various elements of hardware and software may be shared by one or more customers. In a multi-tenant system, a user is typically associated with a particular customer. In one example, a user (e.g., of the client computing device 112) could be an employee of one of a number of (pharmaceutical) companies which are tenants, or customers, of the provider computing system 104.

In some embodiments, the provider computing system 104 may run on a cloud computing platform. Users can access content on the cloud independently by using a virtual machine image or purchasing access to a service maintained by a cloud repository provider.

In some embodiments, the provider computing system 104 may be provided as Software as a Service (“SaaS”) to allow users to access the provider computing system 104 with a thin client.

As shown, the provider computing system 104 may include a network interface 126, a processing circuit 128, a medical product repository 132, a case repository 134, and a rule repository 135. In some embodiments, the provider computing system 104 may include an input/output circuit (e.g., similar to (e.g., the same as) an input/output circuit 180 as will described further herein).

The network interface 126 is structured to establish connections with the partner computing devices 108 and the client computing devices 112 by way of the network 118. The network interface 126 includes program logic (e.g., AS2 Gateway Logic 127) and/or hardware-based components that connect the provider computing system 104 to the network 118. For example, the network interface 126 may include any combination of a wireless network transceiver (e.g., a cellular modem, a broadband modem, a Bluetooth transceiver, a Wi-Fi transceiver, a Li-Fi transceiver, etc.) and/or a wired network transceiver (e.g., an Ethernet transceiver). In some embodiments, the network interface 126 includes the hardware and machine-readable media structured to support communication over multiple channels of data communication (e.g., wireless, Bluetooth, near-field communication (NFC). In some embodiments, the network interface 126 includes cryptography logic and capabilities to establish a secure communications session.

The AS2 gateway logic 127 includes programmable instructions that facilitate communication (transmission and receipt) using the Applicability Statement 2 (AS2) communication protocol (as specified in Request for Comment (RFC) 4130) over the network 118 via the network interface circuit 126. For example, using the AS2 gateway logic 127, the network interface 126 may transmit or receive files (e.g., the source file, a case, etc.) or other data to the partner computing systems 108 or client computing devices 112 using the AS2 Gateway protocol. In other embodiments, the AS2 gateway logic 127 may transmit or receives files using the most updated Applicability Statement communication protocol (e.g., AS3, etc.).

The processing circuit 128, as shown, comprises a memory 136, a processor 140, a case intake and management circuit 142, a medical product verification circuit 144, and a submission management circuit 148. The memory 136 includes one or more memory devices (e.g., RAM, NVRAM, ROM, flash memory, hard disk storage, etc.) that store data and/or computer code for facilitating the various processes described herein. That is, in operation and use, the memory 136 stores at least portions of instructions and data for execution by the processor 140 to control the processing circuit 128. The memory 136 may be or include tangible, non-transient volatile memory and/or non-volatile memory. The processor 140 may be implemented as a general-purpose processor, an application specific integrated circuit (ASIC), one or more field programmable gate array (FPGAs), a digital signal processor (DSP), a group of processing components or other suitable electronic processing components.

As described herein, the case intake and management circuit 142 is structured or configured to receive, generate, store, and manage case datasets. For instance, the case intake and management circuit 142 may be configured or structured to periodically receive or retrieve a source file including adverse event data associated with an adverse event from a trusted source (e.g., one of the partner case repositories 162). In some embodiments, the case intake and management circuit 142 may then match the adverse event data with medical product data of the medical product repository 132, generate a case dataset including case data, and store the case dataset within the case repository 134. In one example, the case intake and management circuit 142 may be an instance of Vault Safety®. In some embodiments, the provider computing system 104 may include multiple case intake and management circuits 140 (e.g., one for each customer, one for each user, etc.).

In some embodiments, when generating the case dataset, the case intake and management circuit 142 may further search the case repository 134 for case datasets which may be a duplicate of the newly generated case. In other embodiments, the case intake and management circuit 142 may output (via the network interface circuit 126) one or more of the case datasets as a specific file type (e.g., E2B (R3) XML file, as a Council for International Organizations of Medical Sciences (CIOMS) II PDF file, etc.) to one or more of the partner computing systems 108 or the client computing devices 112. In other embodiments, the submission management circuit 148 outputs the case datasets and/or the digital documents described herein. In some embodiments, the medical product verification circuit 144 matches the adverse event data of the source file with the medical product data of the medical product repository 132, as will be described further herein. In this regard, the case intake and management circuit 142 may retrieve or receive (e.g., intake) the source file, and then provide the source file to the medical product verification circuit 144 for verification of the specific medical product identified in the source file.

Additionally, the case intake and management circuit 142 may be configured or structured to retrieve cases from the case repository, in response to receiving a request including case criteria (e.g., a medical product of the case, a date of the case, a state of the case (e.g., open, closed, submitted, pending review), a country in which the case took place, etc.) from one of the client computing devices 112.

As described herein, the medical product verification circuit 144 is structured or configured to receive the source file identifying a medical product (or a substance), receive and/or retrieve a medical product verification ruleset including multiple medical product verification rules, and then verify the medical product identified by the source file is the same as one of the customer's or user's medical products (e.g., stored in the medical product repository 132) based on the medical product verification ruleset.

For instance, a source file may be received that includes adverse event data. The adverse event data may include patient data, medical product data, a medical code, and reporter data. For instance, the adverse event data may indicate a patient (e.g., Jane Doe) was hospitalized in the state of Montana for blindness (as indicated by the medical code for blindness) while or after taking 10 MG, orally, of a medical product (which is identified by a trade name or alias (e.g., “Cholecap”) and treated by the reporter (e.g., Dr. Carlson with national provider identifier (NPI) 111111). As a result, medical product verification circuit 144 may search or query the rule repository 135 for a medical product verification ruleset associated with the trade name or alias of the medical product (e.g., “Cholecap”) and return a matching medica product verification ruleset. The ruleset may include multiple medical product verification rules, each medical product verification rule including at least one rule criterion and a specific medical product.

Then, the medical product verification circuit 144 may determine if the medical product matches or fulfills any of the medical product verification rules. For instance, the medical product verification ruleset may include a first medical product verification rule including three rule criteria: a first rule criterion of a dose strength (e.g., 40), a second rule criterion of a dose strength unit (e.g., mg), and a third rule criterion of a dose form (e.g., capsule, gelatin coated). Accordingly, the medical product data may match or fulfill the three rule criteria (e.g., the medical product data may include a dose strength of 40 mg and a dose form of gelatin coated capsule). Accordingly, in response to the medical product data fulfilling the rule criteria of the first medical product verification rule, the medical product verification circuit 144 may verify the specific medical product of the source file as the medical product of the first medical product verification rule.

Additionally, in response to verifying the specific medical product of the source file, the medical product verification circuit 144 may provide the medical product data of the verified medical product to the case intake and management circuit 142. In response, the case intake and management circuit 142 may generate a case dataset including the medical product data and at least a portion of the adverse event data of the source file. In some embodiments, if the medical product of the source file is not verified (e.g., does not fulfill any of the medical product verification rules of the medical product verification ruleset), the medical product verification circuit 144 may generate a notification indicating as such and provide the notification to one of the client computing devices 112 (e.g., via email, via an internal message, via an icon on a user interface, etc.) for display thereon.

The submission management circuit 148 is structured or configured to output or provide the generated case dataset to the one of the partner computing systems 108, in response to generating the case dataset. For instance, using the example above, the submission management circuit 148 may generate an electronic submission associated with the generated case dataset and output the case dataset (e.g., as an E2B (R2 or R3) XML file, as a CIOMS II PDF file, as an email, etc.) to the partner computing system 108 associated with the FDA (e.g., the FDA submissions gateway), in response to the case originating in the USA. In some embodiments, the submission management circuit 148 may output the case dataset using the AS2 communication protocol to the partner computing system 108. In other embodiments, other communication protocols (e.g., file transfer protocol (FTP), email, etc.) may be used to output or transmit the case dataset to the partner computing system 108.

Still referring to FIG. 1, the medical product repository 132 may be repository (e.g., a database) that is structured or configured to receive, store, and manage medical products and medical product data of users or customers. In this regard, the medical product repository 132 may receive, store, and manage medical products including medical product registrations. For example, a customer may have a medical product registered with the FDA (e.g., after applying for and receiving approval) and the EMA. The medical product registration with the FDA may include a medical product name, a list of substance(s) or active ingredient(s), a dose or strength, a route of administration, a marketing status, and/or a national drug code (NDC) identifier. The medical product registration with the EMA may include the same but have a different identifier. Accordingly, one of the client computing devices 112 may provide medical product data including each piece of the FDA medical product registration and the EMA medical product registration to the provider computing system 104 for storage in the medical product repository 132. Accordingly, the medical product repository 132 may receive the medical product data and store the medical product data in association with two specific medical products (e.g., one for the FDA and the USA and one for the EMA and the specific country(s) in Europe). In that regard, each medical product stored in the medical product repository 132 may represent a single medical product registration. In this way, a single substance or group of substances may be represented by multiple medical products in the medical product repository 132 (e.g., a first medical product associated with a first medical product registration for the EMA, a second medical product associated with a second medical product registration for the FDA, a third medical product associated with a third medical product registration for Health Canada, and so on).

Additionally, the medical product repository 132 can be structured according to various database types, such as, relational, hierarchical, network, flat, point-in time, and/or object relational. Further, the medical product repository 132 may include a plurality of nonvolatile/non-transitory storage media such as solid-state storage media, hard disk storage media, virtual storage media, cloud-based storage drives, storage servers, and/or the like.

Likewise, the case repository 134 may be repository (e.g., a database) that is structured or configured to receive, store, and manage case datasets and their respective data (e.g., case data, adverse event data, etc.). For example, the case repository 134 may receive case datasets and related case objects and store the case datasets therein. Then, in response to receiving a query or a request for one or more case datasets (e.g., a query for all cases that include a specific substance), the case repository 134 may provide and/or return the case datasets stored therein that match the query or request. For example, the case repository 134 may receive a query from the medical product verification circuit 144 for all cases that include a specific criteria (e.g., a specific medical product, a specific country of origin, a specific date range). In response, the case repository 134 may determine each case dataset that includes the specific criteria stored therein and return each case dataset. Further, the case repository 134 can be structured according to various database types, such as, relational, hierarchical, network, flat, point-in time, and/or object relational. In some embodiments, the case repository 134 includes a plurality of nonvolatile/non-transitory storage media such as solid-state storage media, hard disk storage media, virtual storage media, cloud-based storage drives, storage servers, and/or the like.

Similarly, the rule repository 135 may be repository (e.g., a database) that is structured or configured to receive, store, and manage medical product verification rulesets and rules of users or customers. In this regard, the rule repository 135 may receive, store, and manage medical product verification rulesets including medical product verification rules. Each medical product verification rule includes a medical product trade name or alias, one or more rule criterion, an order or rank, a target medical product, and the like. For example, a customer may provide or generate a rule set that includes a medical product verification rule set for the trade name “Drug X”. The rule set may include a first medical product verification rule and a second medical product verification rule. Each rule may include the medical product trade name, one or more rule criterion, a unique order (e.g., 1 and 2), and a target medical product. Accordingly, the rule repository 135 may receive the rule set from one of the client computing devices 112 and store it therein. Then, in response to receiving a source file including the trade name, the medical product verification circuit 144 may select the rule from the rule set repository 135 and determine if the medical product data of the source file fulfills any of the medical product verification rules. Accordingly, the rule repository 135 can be structured according to various database types, such as, relational, hierarchical, network, flat, point-in time, and/or object relational. Further, the rule repository 135 may include a plurality of nonvolatile/non-transitory storage media such as solid-state storage media, hard disk storage media, virtual storage media, cloud-based storage drives, storage servers, and/or the like.

While not shown, in some embodiments, the provider computing system 104 may include a separate repository for each data type described herein. For instance, the provider computing system 104 may include the case repository 134, the medical product repository 132, the rule repository 135, a reporter repository (not shown), a health code repository (not shown), a partner repository (for storing electronic addresses, communication protocols, and the like associated with partner computing systems 108) (not shown), and the like.

Still referring to FIG. 1, the partner computing systems 108 may be managed by third-party partners (e.g., the FDA, the EHA, Health Canada, partner company 1, partner company 2, partner computing system xyz, etc.) and can be or include a computing device or system configured to communicate with the provider computing system 104 over the network 118. For instance, the partner computing systems 108 can be a server computer system, a gateway computing system, a laptop computer a desktop computer, and any other network-connected device that can communicate over the network 118. For example, one of the partner computing systems 108 may be the Electronics Submission Gateway (ESG) of the FDA through which one or more E2B XML files may be received from and/or provided to. In another example, one of the partner computing systems 108 may be a laptop computer operated by an employee of a partner company.

In operation, the partner computing systems 108 may communicate with the provider computing system 104 or the client computing device 112 to send and/or receive one or more electronic communications (e.g., case datasets, source files, etc.). For instance, a customer (e.g., pharma company 123) may submit case datasets to the FDA over the ESG of the FDA. Accordingly, the provider computing system 104 may provide case datasets to the first partner computing system associated with the FDA. For instance, the provider computing system 104 (and more specifically the submission management circuit 148) may generate an outbound transmission including one or more case datasets. Then, the provider computing system 104 may output the outbound transmission to the first partner computing system 108.

As shown, each partner computing system 108 includes a network interface 156, a processing circuit 160, and a partner case repository 162. In some embodiments, each partner computing system 108 further includes a key repository (not shown) for storing AS2 keys and certificates.

The network interface 156 is structured to establish connections with the provider computing system 104 and/or the client computing device 112 by way of the network 118. The network interface 156 includes program logic (e.g., AS2 Gateway logic 157) and/or hardware-based components that connect each partner computing system 108 to the network 118. For example, the network interface 156 may include any combination of a wireless network transceiver (e.g., a cellular modem, a broadband modem, a Bluetooth transceiver, a Wi-Fi transceiver, a Li-Fi transceiver, etc.) and/or a wired network transceiver (e.g., an Ethernet transceiver). In some embodiments, the network interface 156 includes the hardware and machine-readable media structured to support communication over multiple channels of data communication (e.g., wireless, Bluetooth, near-field communication (NFC). In some embodiments, the network interface 156 includes cryptography logic and capabilities to establish a secure communications session.

The AS2 gateway logic 157 includes programmable instructions that facilitate communication (transmission and receipt) using the Applicability Statement 2 (AS2) communication protocol (as specified in Request for Comment (RFC) 4130) over the network 118 via the network interface circuit 156. For example, using the AS2 gateway logic 157, the network interface 156 may transmit or receive files (e.g., the source file, a case, etc.) or other data to the provider computing system 104 or client computing devices 112 using the AS2 Gateway protocol. In other embodiments, the AS2 gateway logic 127 may transmit or receives files using the most updated Applicability Statement communication protocol (e.g., AS3, etc.).

The processing circuit 160, as shown, comprises a memory 168 and processor 170. The memory 168 includes one or more memory devices (e.g., RAM, NVRAM, ROM, flash memory, hard disk storage, etc.) that store data and/or computer code for facilitating the various processes described herein. That is, in operation and use, the memory 168 stores at least portions of instructions and data for execution by the processor 170 to control the processing circuit 160. The memory 168 may be or include tangible, non-transient volatile memory and/or non-volatile memory. The processor 170 may be implemented as a general-purpose processor, an application specific integrated circuit (ASIC), one or more field programmable gate array (FPGAs), a digital signal processor (DSP), a group of processing components or other suitable electronic processing components.

The partner case repository 162 may be similar or the same as the case repository 134 and is a repository (e.g., a database, cloud storage, etc.) that is structured or configured to receive, store, and manage case datasets associated with adverse events. For example, one of the partner computing systems 108 may receive a case dataset from the provider computing system 104 and store case dataset in the partner case repository 162. Further, the partner case repository 162 can be structured according to various database types, such as relational, hierarchical, network, flat, point-in time, and/or object relational. In some embodiments, the partner case repository 162 includes a plurality of nonvolatile/non-transitory storage media such as solid-state storage media, hard disk storage media, virtual storage media, cloud-based storage drives, storage servers, and/or the like.

Still referring to FIG. 1, the client computing devices 112 can each be any type of computing device or computing system. For instance, each client computing device 112 can be one or more of a mobile phone, a tablet computer, a laptop computer, a smart watch, a server computer system, or any other internet-connected device. In operation, the client computing devices 112 may communicate and interface with the provider computing system 104 via the network 118 to provide the medical product data for storage therein, medical product verification rulesets, and source files. As shown, each client computing device 112 may include a network interface 176, a processing circuit 178, and the input/output (I/O) circuit 180.

The network interface 176 structured to establish connections with the provider computing system 104 by way of the network 118. The network interface 176 includes program logic and/or hardware-based components that connect the client computing device 112 to the network 118. For example, the network interface 176 may include any combination of a wireless network transceiver (e.g., a cellular modem, a broadband modem, a Bluetooth transceiver, a Wi-Fi transceiver, a Li-Fi transceiver, etc.) and/or a wired network transceiver (e.g., an Ethernet transceiver). In some embodiments, the network interface 176 includes the hardware and machine-readable media structured to support communication over multiple channels of data communication (e.g., wireless, Bluetooth, near-field communication (NFC). In some embodiments, the network interface 176 includes cryptography logic and capabilities to establish a secure communications session.

The processing circuit 178, as shown, comprises a memory 182, a processor 184, and a user interface generation or rendering circuit 186. The memory 182 includes one or more memory devices (e.g., RAM, NVRAM, ROM, flash memory, hard disk storage, etc.) that store data and/or computer code for facilitating the various processes described herein. That is, in operation and use, the memory 182 stores at least portions of instructions and data for execution by the processor 184 to control the processing circuit 178. The memory 182 may be or include tangible, non-transient volatile memory and/or non-volatile memory. The processor 184 may be implemented as a general-purpose processor, an application specific integrated circuit (ASIC), one or more field programmable gate array (FPGAs), a digital signal processor (DSP), a group of processing components or other suitable electronic processing components.

The user interface rendering circuit 186 may be configured to receive a user interface (e.g., a web interface in an HTML file and related files, a downloaded graphical user interface, etc.) from the provider computing system 104 and render the user interface on the client computing device 112 via the I/O circuit 180. In this way, the provider computing system 104 may generate one or more user interfaces and provide the one or more user interfaces to the user interface generation circuit 186 to be rendered on the client computing device 112 (e.g., on a display of the I/O circuit 180 of the client computing device 112).

The I/O circuit 180 is structured to receive communications from and provide communications to the user of the client computing device 112 (e.g., the user). In this regard, the I/O circuit 180 is structured to exchange data with the processing circuit 178 to provide output to the user and to receive input from the user. As a result, the I/O circuit 180 may include a display that may be manipulated by the application. In some embodiments, the I/O circuit 180 may also include a keyboard, a mouse, a joystick, a touch screen, touch areas, soft keys, a microphone, a speaker, a vibration mechanism, a sensor, a RFID scanner, or other input/output devices described herein.

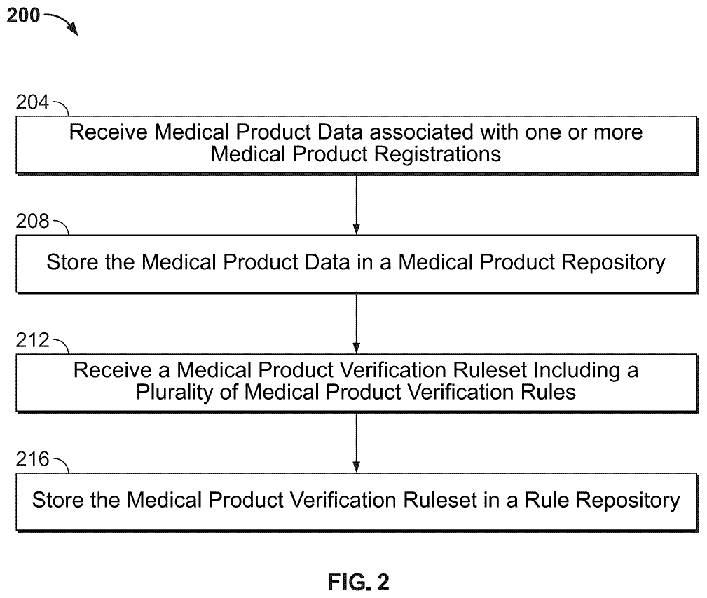

Referring now to FIG. 2, a method 200 of receiving and intaking medical product data and a medical product verification ruleset is shown, according to an example embodiment. Method 200 can be carried out by the system of FIG. 1. More particularly, the method 200 can be carried out by the processing circuit 128 of the provider computing system 104 and through communication with the partner computing systems 108 and the client computing devices 112.

Method 200 commences at step 204 at which the provider computing system 104 receives medical product data associated with one or more medical product registrations. In some embodiments, the medical product data may be received from from one of the client computing devices 112. As described herein, the customer (e.g., via the client computing devices 112) may provide medical product registrations and the associated data to the provider computing system 104 for storage. The medical product data may be used to determine or verify the medical products of received source files are the medical product of the customer and not a different medical product. In one example, a customer may market and sell a medical product (e.g., a drug) that comes in multiple forms and doses (e.g., Cholecap-Pen 40 mg, Cholecap-Pen 80 mg, Cholecap-gelatin capsule 40 mg, and so on). Further, each medical product dose and form may have a unique medical product registration with each health authority (a first medical product registration is for or associated with the FDA and the Cholecap-Pen 40 mg, a second medical product registration is for or associated with the FDA and the Cholecap-Pen 80 mg, a third medical product registration is for or associated with the EMA and the Cholecap-Pen 40 mg, and so on). Accordingly, the customer, via the client computing device 112, may provide registration specific medical product data (e.g., drug identifier number (e.g., NDC, Anatomical Therapeutic Chemical (ATC), etc.), route of administration, dose, country of origin, associated health agency etc.) that identify and define each specific medical product registration. Accordingly, at step 204, the client computing device 112 may provide medical product data associated with one or more medical product registrations to the provider computing system 104. The medical product data may include an NDC, a drug identifier, a route of administration, a dose and/or strength, a country with which the registration is registered/associated, one or more substances included in the medical product, a marketing status, a drug name, the company which holds the marketing authorization, and the like.

Once the provider computing system 104 has received the medical product data, the method 200 proceeds to step 208 at which the provider computing system 104 stores the medical product data in the medical product repository 132. As described herein, the medical product repository 132 may store each medical product by medical product registration such that the same substance or active ingredient (or group thereof) has multiple medical products stored in the medical product repository 132 (e.g., one for the FDA, one for the EMA, one for the Pmda, and so on). In other embodiments, the medical product repository 132 may only store a single medical product for each substance or active ingredient (or group thereof), and each single medical product may include the multiple medical product registrations.

Once the provider computing system 104 has stored the medical product data in the medical product repository 132, the method 200 proceeds to step 212 at which the provider computing system 104 receives a medical product verification ruleset including one or more medical product verification rules. In some embodiments, the medical product verification ruleset is received from one of the client computing devices 112. As described herein, each medical product verification ruleset may be associated with a specific trade name or alias of a medical product (e.g., “Drug X”). Further, each medical product verification rule may include at least one rule criterion (which may include the specific trade name or alias), an order or rank, and a target medical product or medical product registration. For example, each medical product verification rule may identify a specific medical product registration of the received and stored medical product data. For instance, the received medical product data may include or be associated with a first medical product registration (e.g., FDA-Cholecap 40 mg; gelatin capsule; NDC 112211) a second medical product registration (e.g., EMA-Cholecap 40 mg; gelatin capsule; ATC 111221), and a third medical product registration (e.g., FDA-Cholecap 80 mg; gelatin capsule; NDC 112212). Likewise, at step 212, the provider computing system 104 may receive a medical product verification ruleset for the trade name “Cholecap.” The medical product verification ruleset may include a first medical product verification rule, a second medical product verification rule, and a third medical product verification rule. The first medical product verification rule may include multiple rule criteria (e.g., a dose strength of 40, a dose unit of mg, a country of USA, and a route of administration of gelatin capsule) and a target medical product of the first medical product registration. Similarly, the second medical product verification rule may include multiple rule criteria (e.g., a dose strength of 40, a dose unit of mg, a country of France, and a route of administration of gelatin capsule) and a target medical product of the second medical product registration. Further, the third medical product verification rule may include multiple rule criteria (e.g., a dose strength of 80, a dose unit of mg, a country of USA, and a route of administration of gelatin capsule) and a target medical product of the third medical product registration.

Once the provider computing system 104 has received the medical product verification ruleset, the method 200 proceeds to step 216 at which the provider computing system 104 stores the medical product verification ruleset in the rule repository 135. As described herein, the rule repository 135 may store each medical product verification ruleset and medical product verification rule thereof.

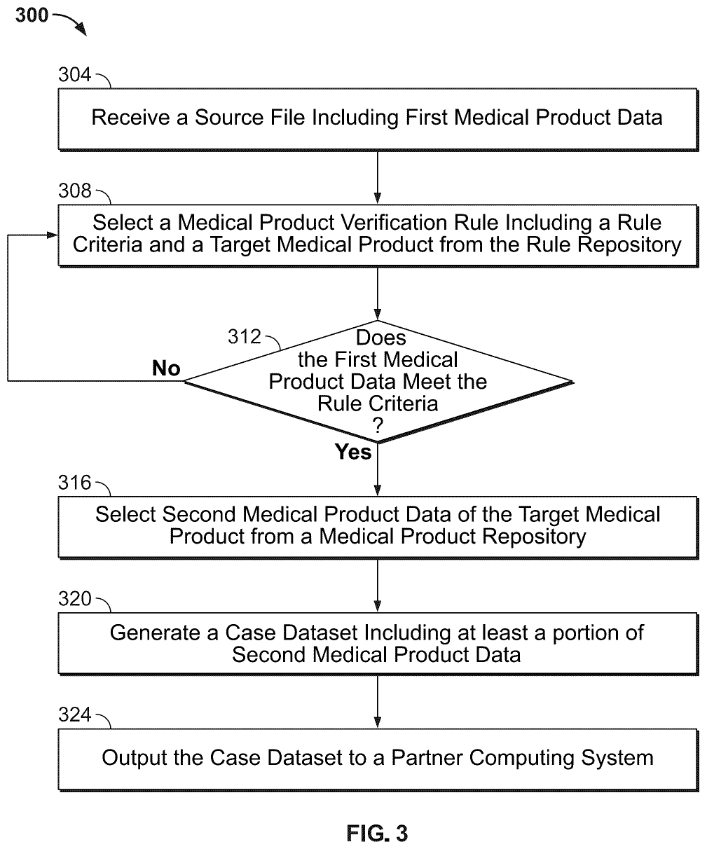

Referring now to FIG. 3, a method 300 of querying and executing a rule is shown, according to an example embodiment. While different overall, it should be understood that any steps or discussion of the method 300 may be applied or included within the method 200, and vice versa, and that such combinations are included within the scope of the present disclosure. For example, the method 200 may include any of the steps 304-324, after or before any steps included in the method 200, and the method 300 may include any of the steps 204-216, after or before any of the steps included in the method 300. In a specific example, the provider computing system 104 may perform the method 200 (e.g., receive medical product data, store the medical product data, receive a medical product verification rule, store the medical product verification rule), and then perform the method 300 (e.g., receive a source file, select the medical product verification rule, generate a case dataset, etc.). Method 300 can be carried out by the system of FIG. 1. More particularly, the method 300 can be carried out by the processing circuit 128 of the provider computing system 104 and through communication with the partner computing systems 108 and the client computing devices 112.

Method 300 commences at step 304 at which the provider computing system 104 receives a source file including first medical product data. The first medical product may include a medical product trade name or alias, and other medical product data (e.g., a dose, a route of administration, a strength, a substance, a lot number, a country of origin, an identifier (e.g., an NDC, an ATC, etc.). In some embodiments, the source file may further include adverse event data associated with one or more adverse events. For instance, the source file may include adverse event information for each adverse event. Further, the source file may be received from one of the client computing devices 112 or one of the partner computing systems 108. In some embodiments, the source file may be an E2B (R2 or R3) XML file received via an AS2 Gateway communication from the one of the partner computing systems 108 or the client computing devices 112. In other embodiments, the source file may be received from one of the partner computing systems 108 via an application programming interface (API) of the provider computing system 104. In other embodiments, the source file may be at least one of a PDF file, an Excel file, an E2B XML file, CSV file, an email, or other file types described herein. The adverse event data may identify or include the first medical product data, study data (e.g., a study identifier), an adverse event term and code (e.g., a MedDRA term and code), reporter data (e.g., a reporter name, a reporter country, a reporter address or contact information (e.g., email, phone number, IP address, FTP address, etc.)), patient data (e.g., patient initials, patient address or contact information), a report type (e.g., spontaneous, from study, from marketed medical product, etc.), a seriousness of the adverse event, and the like.

Once the provider computing system 104 has received the source file, the method 300 proceeds to step 308 at which the provider computing system 104 queries or selects a medical product verification rule from the rule repository 135. As described herein, each medical product verification rule may define the situation or instances in which a specific trade name is to be applied to a target medical product or medical product registration. In this regard, the medical product verification rule may include at least one rule criterion and a target medical product or medical product registration. The rule criterion may be logic statements and pieces of medical product data that are to be compared to the received first medical product data. For instance, the rule criterion may include or identify a country, a dose form, a route of administration, a dose strength, a dose strength unit, a product type (e.g., medical device, biologic, drug, cosmetic, etc.), an organization or company, and the like. Further, the rule criterion may include a logic statement or expression, which is to be used to determine if the first medical product data fulfills the rule criterion (e.g., logical comparator's (e.g., “==” for equal to, “<” for less than, “>” for greater than, “!=” for not equal to, “<=” for less than or equal to), functions (e.g., is_blank( ) is_not_blank( ) is_equal_to( ) range (x,y)), and the like).

In some embodiments, the provider computing system 104 may select the medical product verification rule based on the first medical product data of the source file. For instance, the first medical product data may include a trade name or alias of a medical product. Accordingly, the provider computing system 104 may select the medical product verification rule based on the medical product verification rule including or being associated with the trade name or alias of the medical product.

In some embodiments, prior to step 308, the provider computing system 104 may select a medical product verification ruleset based on the medical product verification ruleset matching or meeting the first medical product data. For instance, as described herein each medical product verification ruleset may be associated with or for a specific medical product trade name of alias (e.g., “Drug X”). Accordingly, in response to the first medical product data including or identifying the specific trade alias, the provider computing system 104 may search or query the rule repository for a medical product verification ruleset associated with or including the specific trade alias. Then, in response to returning a matching medical product verification ruleset, the provider computing system 104 may proceed to step 308 and select a medical product verification rule of the medical product verification ruleset. In some embodiments, each medical product verification ruleset may include an order or a rank, and the provider computing system 104 may first select the medical product verification rule including the order or rank of “1” (i.e., the first rank).

Once the provider computing system 104 has selected the medical product verification rule, the method proceeds to step 312 at which the provider computing system 104 determines if the first medical product data meets or fulfills the rule criteria. To determine if the first medical product data meets or fulfills the rule criteria, the provider computing system 104 may determine if the fields or pieces of medical product data identified in the rule criteria are the same as specified in the rule criteria. For instance, the rule criteria may include a specific dose strength (e.g., 40), a specific dose unit (ml), and a specific route of administration (e.g., intravenous). Accordingly, in response to each of the rule criteria matching or being the same as the respective pieces of first medical product data (e.g., the first medical product includes a specific dose strength of 40, a specific dose unit of ml, and a specific route of administration of intravenous), the provider computing system 104 may determine the rule criteria is met or fulfilled by the first medical product data. In other embodiments, provider computing system 104 may determine the rule criteria is met, in response to a single rule criterion being met or fulfilled by the first medical product data.

In some embodiments, the rule criterion may include a matches_registration( ) function. The matches registration function may indicate the rule criterion is fulfilled if the first medical product data matches the target medical product registration. Accordingly, prior to or at step 312, the provider computing system 104 may query or select the target medical product registration and select the indicated value for comparison to the first medical product data. For instance, a medical product verification rule may include a rule criterion of Dose_Strength (matches_registration( ) and a medical product registration of “Cholecap Registration 1”. Accordingly, prior to or at step 312, the provider computing system 104 may query or search the medical product repository 132 for Cholecap Registration 1. Then, in response to returning the matching registration, the provider computing system 104 may determine if the dose strength of the first medical product data matches or is equal to the dose strength of the matching registration. If they match, the provider computing system 104 may determine the rule criterion is fulfilled.

If, at step 312, the provider computing system 104 determines the first medical product data of the source files does not meet or fulfill the rule criteria, the method 300 may proceed back to step 308 where a different rule (e.g., of the ruleset) is selected from the rule repository 135. In some embodiments, the provider computing system 104 may select the medical product verification rule with the next highest rank or order. For instance, the provider computing system 104 may first select the medical product verification rule with a first ranking (e.g., 1). Then, in response to the first medical product data not fulfilling the rule criteria of the medical product verification rule, the provider computing system 104 may proceed back to step 308 to select the medical product verification rule with a second rank (e.g., “2”). These steps may be repeated until a medical product verification rule is fulfilled by the first medical product data. If the provider computing system 104 proceeds through each medical product verification rule of the medical product verification ruleset without a match or the rule being fulfilled, the method 300 may end.

In comparison, if, at step 312, the provider computing system 104 determines the first medical product data fulfills the rule criteria, the method 300 may proceed to step 316 where the provider computing system 104 queries or selects second medical product data of the target medical product (of the medical product verification rule) from the medical product repository 132. For instance, at step 308, the provider computing system 104 may select a medical product verification rule including rule criteria and a target medical product or medical product registration (e.g., Cholecap registration 1). Then, at step 312, the provider computing system 104 may determine the first medical product data fulfills the rule criteria of the medical product verification rule. Then, at step 316, the provider computing system 104 may select or query second medical product data of the target medical product (e.g., Cholecap registration 1) from the medical product repository 132.

Once the provider computing system has queried or selected the second medical product data of the target medical product from the medical product repository 132, the method 300 proceeds to step 320 where the provider computing system 104 generates a case dataset including at least a portion of the second medical product data. For instance, the case dataset may include the dose, the identifier (e.g., NDC, ATC, etc.), the route of administration, the country of registration, or other data of the second medical product data therein. In some embodiments, the case dataset may include at least a portion of the second medical product data and other data of the source file (e.g., the adverse event data). In some embodiments, the case dataset may include at least a portion of the second medical product data and at least a portion of the first medical product data (e.g., the identifier of the second medical product data and the trade name of the first medical product data). In some embodiments, after generating the case dataset, the provider computing system 104 may store the case dataset in the case repository 134. Once the provider computing system 104 has generated the case dataset, the method 300 proceeds to step 324 at which the provider computing system 104 outputs the case dataset. For instance, the provider computing system 104 may output the case dataset (including the portion of the second medical product data) as an E2B (R2 or R3) XML file to one or more of the partner computing systems 108 via an AS2 Gateway communication. In some embodiments, provider computing system 104 may select the newly generated case dataset and provide it to one of the client computing devices 112. Then, in response to receiving a verification of the case dataset, the provider computing system 104 may output the case dataset to one of the partner computing systems 108.

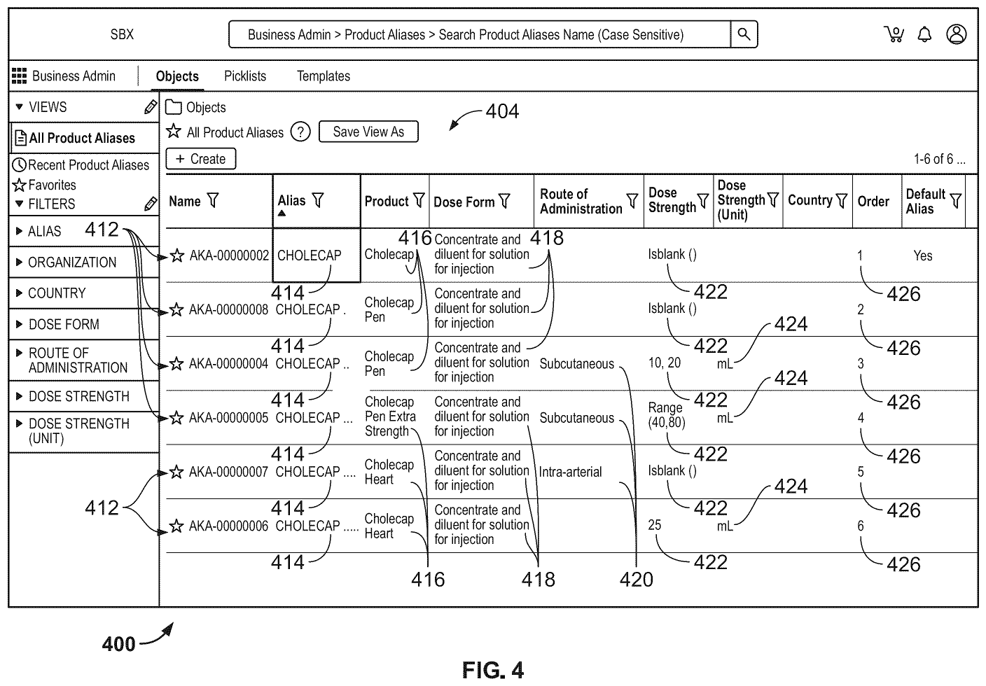

Referring now to FIGS. 4, a user interface shown and displayed to the user of the one or more client computing devices 112 during the methods 200 and/or 300 are shown, according to an example embodiment. As described herein, the user interface of FIG. 4 may be one or more of a web interface generated by the provider computing system 104 and rendered by each of the client computing devices 112 as part of a web application or a graphical user interface downloaded and generated by each of the client computing devices 112 as part of a software application (e.g., a mobile application, etc.). Further, the user interface of FIG. 4 provides for communication between the user) and the provider computing system 104 via the respective client computing device 112 (specifically via the I/O circuit 180, respectfully). Through interaction with the user interface, the user may provide user input, feedback, and other data requested by the provider computing system 104. In this regard, it should be understood that each interaction described herein by the user with the user interface of FIG. 4 may be provided to one or more of the client computing devices 112 and then transmitted to the provider computing system 104 and that each action described herein as occurring to the respective client computing device 112 (e.g., navigating to a certain page, generating a popup, etc.) may be performed by the provider computing system 104.

Referring now to FIG. 4, a medical product verification rule page 400 which can be displayed on a display the I/O circuit 180 of the client computing device 112, is shown. In general, the medical product verification rule page 400 provides the user an interface to setup, modify, and manage the medical product verification rules of a specific customer (e.g., of a medical product verification ruleset). As shown, the medical product verification rule page 400 includes a rule listing 404. To render or generate the medical product verification rule page 400 on the client computing device 112, the provider computing system 104 may provide the medical product verification rules (e.g., of a specific medical product verification ruleset) and associated data to the client computing device 112. In this regard, it should be understood that each of the sections, fields, or buttons of the medical product verification rule page 400 may be or included in the medical product verification rules and rulesets described herein.

The rule listing 404 provides the user of the respective client computing device 112 with an interface to set, manage, and update the medical product verification rules of the medical product verification rule page 400. As shown, the rule listing 404 includes multiple medical product rule representations 412. Each medical product rule representations 412 represents or is associated with a specific medical product rule and includes an alias or trade name field 414, a target medical product field 416, multiple rule criterion fields (including the dose form field 418, the route of administration field 420, the dose strength field 422, the dose strength unit field 424, and the country field (not shown)), and an order or rank field 426. In some embodiments, each medical product rule representations 412 further includes a medical product ruleset field (not shown) including the parent medical product ruleset.

The alias or trade name field 414 may be a field through which the user of the respective client computing device 112 can review and set the alias or trade name of the medical product verification rule. Likewise, the target medical product field 416 may be a field through which the user of the respective client computing device 112 can review and set the target medical product of the medical product verification rule. In some embodiments, the target medical product field 416 may be a selectable link that, when selected, navigates the client computing device 112 to a product page (not shown).

The rule criterion fields may each be fields through which the user of the respective client computing device 112 can review and set the rule criteria of the medical product verification rule. For instance, via the rule criterion fields, the user may set a data field (e.g., route of administration, dose strength, dose strength unit, etc.) and a function associated with the data field (e.g., range function, target medical product function, etc.). Likewise, the order or rank field 426 may be a field through which the user of the respective client computing device 112 can review and set the order or rank of the medical product verification rule. In some embodiments, each medical product rule representation 412 may each include a unique order or rank in the rank field 426.

The embodiments described herein have been described with reference to the drawings. The drawings illustrate certain details of specific embodiments that implement the systems, methods, and programs described herein. However, describing the embodiments with drawings should not be construed as imposing on the disclosure any limitations that may be present in the drawings.

It should be understood that no claim element herein is to be construed under the provision of 35 U.S.C § 112(f), unless the element is expressly recited using the phrase “means for.”

As used herein, the term “circuit” may include hardware structured to execute the functions described herein. In some embodiments, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some embodiments, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR), resistors, multiplexors, registers, capacitors, inductors, diodes, wiring, and so on.

The “circuit” may also include one or more processors communicably coupled to one or more memory or memory devices. In this regard, the one or more processors may execute instructions stored in the memory or may execute instructions otherwise accessible to the one or more processors. In some embodiments, the one or more processors may be embodied in various ways. The one or more processors may be constructed in a manner sufficient to perform at least the operations described herein. In some embodiments, the one or more processors may be shared by multiple circuits (e.g., circuit A and circuit B may comprise or otherwise share the same processor which, in some example embodiments, may execute instructions stored, or otherwise accessed, via different areas of memory). Alternatively or additionally, the one or more processors may be structured to perform or otherwise execute certain operations independent of one or more co-processors. In other embodiments, two or more processors may be coupled via a bus to enable independent, parallel, pipelined, or multi-threaded instruction execution. Each processor may be implemented as one or more general purpose processors, application specific integrated circuits (ASICs), field programmable gate arrays (FPGAs), digital signal processors (DSPs), or other suitable electronic data processing components structured to execute instructions provided by the memory. The one or more processors may take the form of a single core processor, a multi-core processor (e.g., dual core, quad core, etc.), microprocessor, etc. In some embodiments, the one or more processors may be external to the apparatus. For example, the one or more processors may be a remote processor (e.g., a cloud-based processor). Alternatively or additionally, the one or more processors may be internal and/or local to the apparatus. In this regard, a circuit or components thereof may be disposed locally (e.g., as part of a local server, a local computing system) or remotely (e.g., as part of a remote server such as a cloud-based server). To that end, a “circuit” as described herein may include components that are distributed across one or more locations. Further, each of the circuits described herein may be distributed across one or more locations (e.g., each as part of one or more remote servers).

An example system for implementing the overall system or portions of the embodiments might include a general-purpose computing device in the form of computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile storage media may take the form of ROM, flash memory (e.g., flash memory such as NAND, 3D NAND, NOR, 3D NOR), EEPROM, MRAM, magnetic storage, hard disks, optical disks, etc. Combinations of the above are also included within the scope of machine-readable media. In this regard, machine-executable instructions comprise, for example, instructions and data which cause a general-purpose computer, special purpose computer, or special purpose processing machine to perform a certain function or group of functions. Each respective memory device may be operable to maintain or otherwise store data relating to the operations performed by one or more associated circuits, including processor instructions and related data (e.g., database components, object code components, script components), in accordance with the example embodiments described herein.

It should also be noted that the term “input devices,” as described herein, may include any type of input device including, but not limited to, a keyboard, a keypad, a mouse, a joystick, or other input devices performing a similar function. Comparatively, the term “output device,” as described herein, may include any type of output device including, but not limited to, a computer monitor, printer, facsimile machine, or other output devices performing a similar function.

It should be noted that the term “field,” as described herein may include any form of an input field through which the user interfaces shown and described may receive input from a user of a computing device. For instance, the term “field” may include a text field, a drop-down box and selectable options, a list box, a lookup box, a search bar, an icon, one or more checkboxes, one or more radio buttons, a button, a toggle, a date field, a slider, and the like. Further, each “field” may include and/or receive data that may be associated with a data object as described herein.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied. The order or sequence of any element or apparatus may be varied or substituted according to alternative embodiments. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principles of the disclosure and its practical application to enable one skilled in the art to utilize the various embodiments and with various modifications as are suited to the particular use contemplated. Other substitutions, modifications, changes, and omissions may be made in the design, operating conditions and embodiment of the embodiments without departing from the scope of the present disclosure as expressed in the appended claim.

What is claimed is:

1. A method for querying and executing a second rule of a first database, the method comprising:
receiving, by a provider computing system, a source file including first medical product data;
querying, by the provider computing system, a first database of the provider computing system to select a first rule,
wherein the first rule includes at least one first rule criterion;
determining, by the provider computing system, the first medical product data of the source file does not fulfill the at least one first rule criterion;
querying, by the provider computing system and in response to the first medical product data of the source file not fulfilling the at least one first rule criterion, the first database of the provider computing system to select the second rule,
wherein the second rule includes at least one second rule criterion and a target medical product;
determining, by the provider computing system, the first medical product data of the source file fulfills the at least one second rule criterion;
querying, by the provider computing system and in response to the first medical product data of the source file fulfilling the at least one second rule criterion, a second database of the provider computing system to select second medical product data associated with the target medical product;
generating, by the provider computing system, a case dataset including at least a portion of the second medical product data; and
outputting, by the network interface, the case dataset.

2. The method of claim 1, wherein the at least one first rule criterion and the at least one second rule criterion each include at least one of: a dose strength, a dose strength unit, a country, or a route of administration.

3. The method of claim 2, wherein the at least one first rule criterion includes a dose strength and a dose strength unit, wherein the first medical product data includes a dose strength and a dose strength unit, and wherein determining the first medical product data of the source file does not fulfill the at least one first rule criterion comprises:
determining, by the provider computing system, the first medical product data of the source file does not fulfill the at least one first rule criterion in response to:
i) the dose strength of the at least one first rule criterion not being the same as the dose strength of the first medical product data; or
ii) the dose strength unit of the at least one first rule criterion not being the same as the dose strength unit of the first medical product data.

4. The method of claim 3, wherein the at least one second rule criterion includes a dose strength and a dose strength unit, and wherein determining the first medical product data of the source file fulfills the at least one second rule criterion comprises:
determining, by the provider computing system, the first medical product data of the source file fulfills the at least one second rule criterion in response to:
i) the dose strength of the at least one second rule criterion being the same as the dose strength of the first medical product data; and
ii) the dose strength unit of the at least one second rule criterion being the same as the dose strength unit of the first medical product data.

5. The method of claim 1, wherein the at least one second rule criterion includes a first function associated with the target medical product, and wherein determining the first medical product data of the source file fulfills the at least one second rule criterion comprises:
querying, by the provider computing system and in response to the at least one second rule criterion including the first function, the second database of the provider computing system to select the second medical product data associated with the target medical product; and
determining, by the provider computing system, the first medical product data of the source file fulfills the at least one second rule criterion in response to at least a portion of the second medical product data being the same as at least a portion of the first medical product data.

6. The method of claim 5, wherein the at least one second rule criterion includes a dose strength and a first function associated with the target medical product, wherein the first medical product data includes a dose strength, wherein the second medical product data includes a dose strength, and wherein determining the first medical product data of the source file fulfills the at least one second rule criterion comprises:
determining, by the provider computing system, the first medical product data of the source file fulfills the at least one second rule criterion in response to the dose strength of the second medical product data being the same as the dose strength of the first medical product data.

7. The method of claim 1, wherein the at least one second rule criterion includes a first function associated with a range between a first value and a second value, wherein the first medical product data includes a dose strength, and wherein determining the first medical product data of the source file fulfills the at least one second rule criterion comprises:
determining, by the provider computing system, the first medical product data of the source file fulfills the at least one second rule criterion in response to the dose strength of the first medical product data being between the first value and the second value of the first function.

8. The method of claim 1, wherein the first medical product data includes a medical product alias, wherein querying the first database of the provider computing system to select the first rule is based on the first rule including the medical product alias, and wherein querying the first database of the provider computing system to select the second rule is based on the second rule including the medical product alias.

9. The method of claim 1, wherein the source file is an E2B XML file, and wherein the case dataset is output as an E2B XML file.

10. The method of claim 1, wherein the first rule and the second rule each include a rank, wherein the first rule is selected based on the rank of the first rule being a first rank, and wherein the second rule is selected based on the rank of the second rule being a second rank.

11. A method for querying and executing a first rule of a first database, the method comprising:
receiving, by a provider computing system, a source file including first medical product data,
wherein the first medical product data includes a medical product alias;
querying, by the provider computing system, a first database of the provider computing system to select a ruleset in response to the ruleset including the medical product alias,
wherein the ruleset includes at least the first rule and a second rule, and wherein the first rule includes at least one rule criterion and a target medical product;
determining, by the provider computing system, the first medical product data of the source file fulfills the at least one rule criterion;
querying, by the provider computing system and in response to the first medical product data of the source file fulfilling the at least one rule criterion, a second database of the provider computing system to select second medical product data associated with the target medical product;
generating, by the provider computing system, a case dataset including at least a portion of the second medical product data; and
outputting, by the network interface, the case dataset.

12. The method of claim 11, wherein the at least one rule criterion includes at least one of:
a dose strength, a dose strength unit, a country, or a route of administration.

13. The method of claim 12, wherein the at least one rule criterion includes a dose strength and a dose strength unit, and wherein determining the first medical product data of the source file fulfills the at least one rule criterion comprises:
determining, by the provider computing system, the first medical product data of the source file fulfills the at least one rule criterion in response to:
i) the dose strength of the at least one rule criterion being the same as the dose strength of the first medical product data; and
ii) the dose strength unit of the at least one rule criterion being the same as the dose strength unit of the first medical product data.

14. The method of claim 11, wherein the at least one rule criterion includes a first function associated with the target medical product, and wherein determining the first medical product data of the source file fulfills the at least one rule criterion comprises:
querying, by the provider computing system and in response to the at least one rule criterion including the first function, the second database of the provider computing system to select the second medical product data associated with the target medical product; and
determining, by the provider computing system, the first medical product data of the source file fulfills the at least one rule criterion in response to at least a portion of the second medical product data being the same as at least a portion of the first medical product data.

15. The method of claim 14, wherein the at least one rule criterion includes a dose strength and a first function associated with the target medical product, wherein the first medical product data includes a dose strength, wherein the second medical product data includes a dose strength, and wherein determining the first medical product data of the source file fulfills the at least one rule criterion comprises:
determining, by the provider computing system, the first medical product data of the source file fulfills the at least one rule criterion in response to the dose strength of the second medical product data being the same as the dose strength of the first medical product data.

16. The method of claim 11, wherein the at least one rule criterion includes a first function associated with a range between a first value and a second value, wherein the first medical product data includes a dose strength, and wherein determining the first medical product data of the source file fulfills the at least one rule criterion comprises:
determining, by the provider computing system, the first medical product data of the source file fulfills the at least one rule criterion in response to the dose strength of the first medical product data being between the first value and the second value of the first function.

17. The method of claim 11, wherein the source file is an E2B XML file, and wherein the case dataset is output as an E2B XML file.

18. The method of claim 11, wherein the first rule and the second rule each include a rank, and wherein the provider computing system determines the first medical product data of the source file fulfills the at least one rule criterion, in response to the rank of the first rule being a first rank.

19. The method of claim 11, wherein the at least one rule criterion is at least one first rule criterion, wherein the second rule includes at least one second rule criterion, and wherein the method further comprises:
determining, by the provider computing system, the first medical product data of the source file does not fulfill the at least one second rule criterion; and
determining, by the provider computing system and in response to the first medical product data of the source file not fulfilling the at least one second rule criterion, the first medical product data of the source file fulfills the at least one rule criterion.

20. The method of claim 11, wherein the second medical product data is associated with a medical product registration for a specific health agency.
